(19) United States
(12) Patent Application Publication
(10) Pub. No.: US 2013/0144149 A1
(43) Pub. Date: Jun. 6, 2013

(54) PHOTOACOUSTIC IMAGING APPARATUS, PHOTOACOUSTIC SENSING STRUCTURE, AND METHOD OF CAPTURING PHOTOACOUSTIC IMAGE

(71) Applicants: Shih-Bin Luo, Hsinchu County (TW); De-Yi Chiou, New Taipei City (TW); Hsiu-Hsiang Chen, Hsinchu County (TW); Wan-Ting Tien, Chiayi City (TW)

(72) Inventors: Shih-Bin Luo, Hsinchu County (TW); De-Yi Chiou, New Taipei City (TW); Hsiu-Hsiang Chen, Hsinchu County (TW); Wan-Ting Tien, Chiayi City (TW)

(73) Assignee: INDUSTRIAL TECHNOLOGY RESEARCH INSTITUTE, Hsinchu (TW)

(21) Appl. No.: 13/338,267

(22) Filed: Dec. 28, 2011

(30) Foreign Application Priority Data
Dec. 5, 2011 (TW) 100144640

(51) Int. Cl. A61B 6/00

(52) U.S. Cl. 600/407

(57) ABSTRACT

A photoacoustic imaging apparatus for detecting a photoacoustic image of an object, a photoacoustic sensing structure, and a photoacoustic image capturing method are provided. The photoacoustic imaging apparatus includes an electromagnetic wave source for emitting an electromagnetic wave, a first electromagnetic wave transmissible substrate disposed on a transmission path of the electromagnetic wave, electromagnetic wave transmitting needles disposed on the first electromagnetic wave transmissible substrate, and an ultrasonic sensor disposed at one side of the object. The electromagnetic wave transmitting needles can be inserted into the object. The electromagnetic wave is transmitted to at least a part of the electromagnetic wave transmitting needles through the first electromagnetic wave transmissible substrate and to the inside of the object through at least the part of the electromagnetic wave transmitting needles. The inside of the object generates an ultrasonic wave in response to the electromagnetic wave. The ultrasonic sensor detects the ultrasonic wave.

### CROSS-REFERENCE TO RELATED APPLICATION

This application claims the priority benefit of Taiwan application serial no. 100144640, filed on Dec. 5, 2011. The entirety of the above-mentioned patent application is hereby incorporated by reference herein and made a part of this specification.

### BACKGROUND

1. Technical Field

The disclosure relates to a photoacoustic imaging apparatus.

2. Related Art

When a tissue (for example, a living tissue) is irradiated by an electromagnetic wave, the tissue absorbs electromagnetic energy, converts a portion of the electromagnetic energy into acoustic energy, and transmits the acoustic energy as an acoustic wave. Such an effect is referred to as photoacoustic effect. Photoacoustic effect is usually applied to internal imaging of living tissue or sample analysis. For example, photoacoustic effect can be applied to the detection of skin cancer.

Generally speaking, a photoacoustic imaging apparatus includes at least an ultrasonic sensor and an electromagnetic wave source. After a living tissue region is irradiated by using an electromagnetic wave, the living tissue region generates and emits a photoacoustic signal, and the ultrasonic sensor receives the photoacoustic signal to determine imaging characteristics of the living tissue region. However, in a conventional technique, the electromagnetic wave may be reflected or absorbed by other living tissues when it is transmitted to the inside of the living tissue region, so that the quality of the photoacoustic image may be affected. For example, when a photoacoustic imaging apparatus is applied to the detection of melanomas in a conventional technique, since melanomas may grow in the tissue under the epidermis of human skin, the electromagnetic wave is reflected or absorbed by non-uniform tissues (for example, cells, collagenous fibers, or interstitial fluid) in the epidermis. As a result, the melanomas under the epidermis cannot be successfully detected.

### SUMMARY

According to an embodiment of the disclosure, a photoacoustic imaging apparatus for detecting a photoacoustic image of an object is provided. The photoacoustic imaging apparatus includes an electromagnetic wave source capable of emitting an electromagnetic wave, a first electromagnetic wave transmissible substrate disposed on a transmission path of the electromagnetic wave, a plurality of electromagnetic wave transmitting needles disposed on the first electromagnetic wave transmissible substrate, and an ultrasonic sensor disposed at one side of the object. The electromagnetic wave transmitting needles are suitable for being inserted into the object. The electromagnetic wave is transmitted to at least a part of the electromagnetic wave transmitting needles through the first electromagnetic wave transmissible substrate. The electromagnetic wave is transmitted to the inside of the object through at least the part of the electromagnetic wave transmitting needles. The inside of the object generates an ultrasonic wave in response to the electromagnetic wave. The ultrasonic sensor detects the ultrasonic wave.

According to an embodiment of the disclosure, a photoacoustic sensing structure suitable for guiding an electromagnetic wave to the inside of an object to receive an ultrasonic wave generated by the inside of the object in response to the electromagnetic wave is provided. The photoacoustic sensing structure includes a first electromagnetic wave transmissible substrate, a plurality of electromagnetic wave transmitting needles, and an ultrasonic sensor. The first electromagnetic wave transmissible substrate is disposed on a transmission path of the electromagnetic wave. The electromagnetic wave transmitting needles are disposed on the first electromagnetic wave transmissible substrate and are suitable for being inserted into the object. The electromagnetic wave is transmitted to at least a part of the electromagnetic wave transmitting needles through the first electromagnetic wave transmissible substrate. The electromagnetic wave is transmitted to the inside of the object through at least the part of the electromagnetic wave transmitting needles. The ultrasonic sensor is disposed at one side of the object. The inside of the object generates an ultrasonic wave in response to the electromagnetic wave. The ultrasonic sensor detects the ultrasonic wave.

According to an embodiment of the disclosure, a method of capturing a photoacoustic image is provided. The method includes following steps. An object is provided. A first electromagnetic wave transmissible substrate and a plurality of electromagnetic wave transmitting needles disposed on the first electromagnetic wave transmissible substrate are provided. The first electromagnetic wave transmissible substrate is laid on the object, and the electromagnetic wave transmitting needles are inserted into the object. An electromagnetic wave is transmitted to the inside of the object through the first electromagnetic wave transmissible substrate and at least a part of the electromagnetic wave transmitting needles. The inside of the object generates an ultrasonic wave in response to the electromagnetic wave. The ultrasonic wave is detected.

Several exemplary embodiments accompanied with figures are described in detail below to further describe the disclosure in details.

### BRIEF DESCRIPTION OF THE DRAWINGS

The accompanying drawings are included to provide further understanding, and are incorporated in and constitute a part of this specification. The drawings illustrate exemplary embodiments and, together with the description, serve to explain the principles of the disclosure.

FIG. 1 is a cross-sectional view of a photoacoustic imaging apparatus according to a first embodiment of the disclosure.

FIG. 2 is a top view of the photoacoustic imaging apparatus in FIG. 1.

FIG. 3 is a cross-sectional view of a photoacoustic imaging apparatus according to another embodiment of the disclosure.

FIG. 4 is a cross-sectional view of a photoacoustic imaging apparatus according to yet another embodiment of the disclosure.

FIG. 5 is a cross-sectional view of a photoacoustic imaging apparatus according to still another embodiment of the disclosure.

FIG. 6 is a partial cross-sectional view of an ultrasonic sensor in FIG. 1.

FIG. 7 and FIG. 8 illustrate transmittances of human skin with respect to electromagnetic waves of different wavelengths.

FIG. 9 is a cross-sectional view of a photoacoustic imaging apparatus according to a second embodiment of the disclosure.

FIG. 10A˜FIG. 10F illustrates a process of integrating electromagnetic wave transmitting needles and an ultrasonic sensor onto a first electromagnetic wave transmissible substrate.

FIG. 11 is a cross-sectional view of a photoacoustic imaging apparatus according to a third embodiment of the disclosure.

FIG. 12 is a top view of the photoacoustic imaging apparatus in FIG. 11.

FIG. 13 is a cross-sectional view of a photoacoustic imaging apparatus according to a fourth embodiment of the disclosure.

### DETAILED DESCRIPTION OF DISCLOSED EMBODIMENTS

### First Embodiment

### Photoacoustic Imaging Apparatus

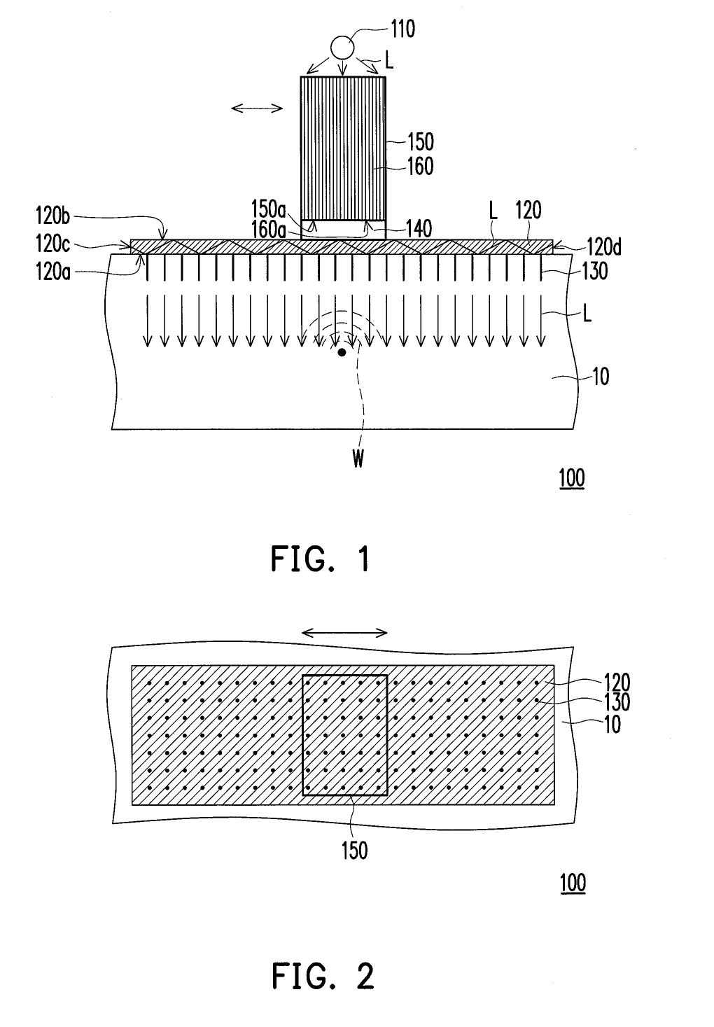

FIG. 1 is a cross-sectional view of a photoacoustic imaging apparatus according to the first embodiment of the disclosure. FIG. 2 is a top view of the photoacoustic imaging apparatus in FIG. 1. Referring to FIG. 1 and FIG. 2, the photoacoustic imaging apparatus 100 in the present embodiment is suitable for detecting an object 10. In the present embodiment, the object 10 is living tissue or inorganic tissue. For example, the object 10 is human skin. The photoacoustic imaging apparatus 100 in the present embodiment includes an electromagnetic wave source 110, a first electromagnetic wave transmissible substrate 120, a plurality of electromagnetic wave transmitting needles 130, and an ultrasonic sensor 140. The first electromagnetic wave transmissible substrate 120, the electromagnetic wave transmitting needles 130, and an ultrasonic sensor 140 constitute a photoacoustic sensing structure.

The electromagnetic wave source 110 in the present embodiment is capable of emitting an electromagnetic wave L. The electromagnetic wave source 110 in the present embodiment may be a laser generator, wherein the laser generator may be a diode laser generator, a solid laser generator, a gas laser generator, or a dye laser generator. In the present embodiment, the wavelength of the electromagnetic wave L is determined to allow the object 10 to have the highest transmittance. For example, in the present embodiment, the wavelength of the electromagnetic wave L falls within a range of 10 nm to 2400 nm.

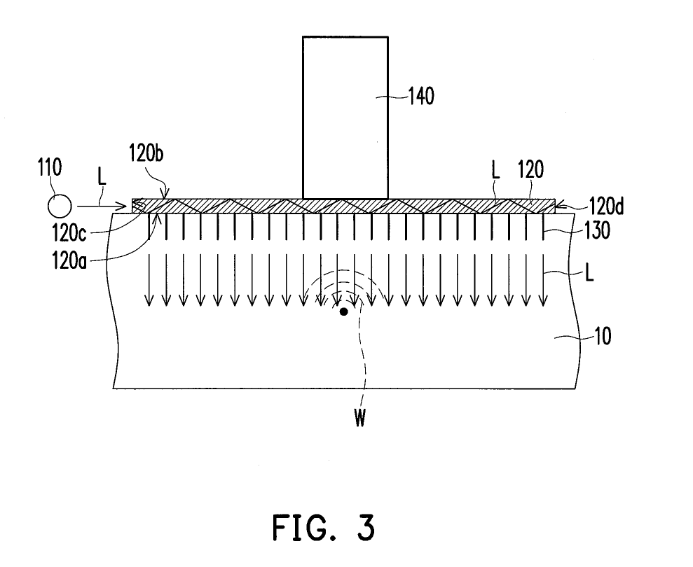

In the present embodiment, the first electromagnetic wave transmissible substrate 120 is disposed on the transmission path of the electromagnetic wave L. In the present embodiment, besides being transmissible to electromagnetic wave, the first electromagnetic wave transmissible substrate 120 is further capable of guiding the electromagnetic wave L to the electromagnetic wave transmitting needles 130. To be specific, in the present embodiment, the first electromagnetic wave transmissible substrate 120 includes a first surface 120a, a second surface 120b opposite to the first surface 120a, and electromagnetic wave incident surfaces 120c and 120d, wherein the second surface 120b may be an electromagnetic wave incident surface. The electromagnetic wave transmitting needles 130 is disposed on the first surface 120a. The electromagnetic wave L enters the first electromagnetic wave transmissible substrate 120 through the electromagnetic wave incident surfaces 120c and 120d, the second surface 120b, or a combination of foregoing surfaces. The electromagnetic wave L is transmitted into the electromagnetic wave transmitting needles 130 dispersedly through the first surface 120a under the guidance of the first electromagnetic wave transmissible substrate 120. However, the disclosure is not limited thereto. FIG. 3 is a cross-sectional view of a photoacoustic imaging apparatus according to another embodiment of the disclosure. Referring to FIG. 3, in this embodiment, the electromagnetic wave L enters the first electromagnetic wave transmissible substrate 120 through the electromagnetic wave incident surface 120c (or 120d).

Additionally, the first electromagnetic wave transmissible substrate 120 in the present embodiment is made of a soft material. In other words, the first electromagnetic wave transmissible substrate 120 in the present embodiment is a flexible substrate, wherein the material of the flexible substrate may be polyethylene terephthalate (PET), polyimide, or any other suitable material. Because the first electromagnetic wave transmissible substrate 120 in the present embodiment is made of a soft material, when a user is about to insert the electromagnetic wave transmitting needles 130 fixed on the first electromagnetic wave transmissible substrate 120 into the object 10, the first electromagnetic wave transmissible substrate 120 can be closely laid on the surface of the object 10 along the contour of the object 10 so that the electromagnetic wave transmitting needles 130 can be nicely inserted into the object 10.

In the present embodiment, the electromagnetic wave transmitting needles 130 are disposed on the first electromagnetic wave transmissible substrate 120. To be specific, as shown in FIG. 2, the electromagnetic wave transmitting needles 130 in the present embodiment are arranged on the first electromagnetic wave transmissible substrate 120 as an array. The electromagnetic wave transmitting needles 130 can be inserted into the object 10. The electromagnetic wave L can be transmitted into at least a part of the electromagnetic wave transmitting needles 130 through the first electromagnetic wave transmissible substrate 120. Besides, the electromagnetic wave L can be transmitted to the inside of the object 10 through at least the part of the electromagnetic wave transmitting needles 130.

It should be mentioned that through the electromagnetic wave transmitting needles 130 in the present embodiment, the possibility that the electromagnetic wave L is absorbed or reflected by the object 10 when it is transmitted to the inside of the object 10 is greatly reduced, so that the electromagnetic wave L can be effectively transmitted to the inside of the object 10. Accordingly, the quality of the photoacoustic image captured by the photoacoustic imaging apparatus 100 in the present embodiment can be considerably improved.

Because the electromagnetic wave transmitting needles 130 in the present embodiment are suitable for being inserted into living tissue, the material of the electromagnetic wave transmitting needles 130 should be biocompatible. For example, the material of the electromagnetic wave transmitting needles 130 may be chitosan or any other suitable material. In the present embodiment, the length and diameter of the electromagnetic wave transmitting needles 130 can be adjusted according to the actual requirement. For example, if the object to be detected is human skin, the length of the electromagnetic wave transmitting needles 130 falls within a range of 100 μm to 1000 μm so that epidermis or dermis of the human skin can be observed. The diameter of the electromagnetic wave transmitting needles 130 falls within a range of 20 μm to 300 μm so that the electromagnetic wave transmitting needles 130 can be easily inserted into human skin within causing too much discomfort.

In the present embodiment, the ultrasonic sensor 140 is disposed at one side of the object 10. The inside of the object 10 generates an ultrasonic wave W in response to the electromagnetic wave L. The ultrasonic sensor 140 detects the ultrasonic wave W generated by the inside of the object 10. To be specific, when the electromagnetic wave L is transmitted to the inside of the object 10, the inside of the object 10 produces thermal expansion and contraction due to the absorption of the electromagnetic wave and accordingly generates the ultrasonic wave W. A signal generated by the ultrasonic sensor 140 after it receives the ultrasonic wave W is appropriately processed so that a photoacoustic image of the inside of the object 10 is obtained.

It should be noted that in the present embodiment, because the ultrasonic wave W needs to run from the object 10 to the ultrasonic sensor 140 through the first electromagnetic wave transmissible substrate 120, the physical characteristic of the first electromagnetic wave transmissible substrate 120 needs to be specially designed so that the ultrasonic wave W won't attenuate when it passes through the object 10 and the first electromagnetic wave transmissible substrate 120. To be specific, in the present embodiment, the first electromagnetic wave transmissible substrate 120 has an ultrasonic wave impedance matching characteristic with respect to the object 10.

The photoacoustic imaging apparatus 100 in the present embodiment further includes a probe 150. The electromagnetic wave L is transmitted to the first electromagnetic wave transmissible substrate 120 through the probe 150. The probe 150 has an opening 150a. The electromagnetic wave L is transmitted to the first electromagnetic wave transmissible substrate 120 through the opening 150a. The opening 150a may be in a linear shape, a circular shape, an array-like shape, or any other suitable shape. The photoacoustic imaging apparatus 100 in the present embodiment further includes an electromagnetic wave transmitter 160 (for example, a fiber bundle) disposed in the probe 150. The electromagnetic wave transmitter 160 transmits the electromagnetic wave L emitted by the electromagnetic wave source 110 to the first electromagnetic wave transmissible substrate 120.

The ultrasonic sensor 140 in the present embodiment is suitable for being passed through by the electromagnetic wave L. To be specific, in the present embodiment, the transmittance of the ultrasonic sensor 140 with respect to the electromagnetic wave L is greater than 60%. In the present embodiment, the electromagnetic wave transmitter 160 has an electromagnetic wave exit surface 160a. In the present embodiment, the ultrasonic sensor 140 is disposed on the electromagnetic wave exit surface 160a. The electromagnetic wave L transmitted in the electromagnetic wave transmitter 160 sequentially passes through the electromagnetic wave exit surface 160a, the ultrasonic sensor 140, and the first electromagnetic wave transmissible substrate 120 and eventually enters the object 10 through the electromagnetic wave transmitting needles 130.

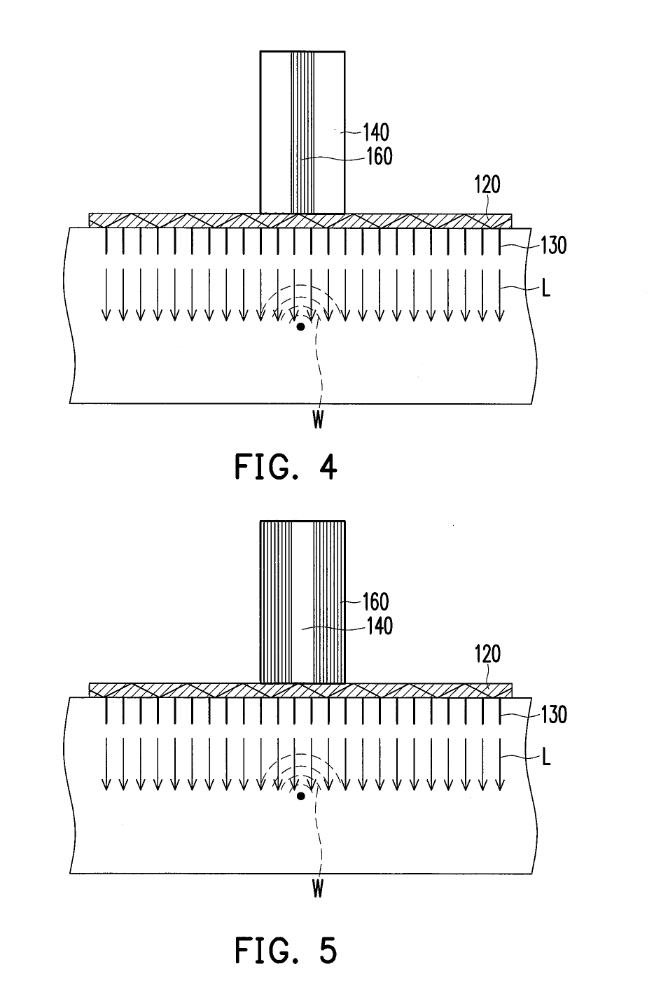

However, the disclosure is not limited to foregoing description, and in other embodiments, the ultrasonic sensor 140 may also be disposed in other ways. FIG. 4 is a cross-sectional view of a photoacoustic imaging apparatus according to yet another embodiment of the disclosure. Referring to FIG. 4, in the present embodiment, the ultrasonic sensor 140 is disposed at the periphery of the electromagnetic wave transmitter 160. To be specific, in the present embodiment, the ultrasonic sensor 140 surrounds the electromagnetic wave transmitter 160. FIG. 5 is a cross-sectional view of a photoacoustic imaging apparatus according to still another embodiment of the disclosure. Referring to FIG. 5, in the present embodiment, the ultrasonic sensor 140 is surrounded by the electromagnetic wave transmitter 160.

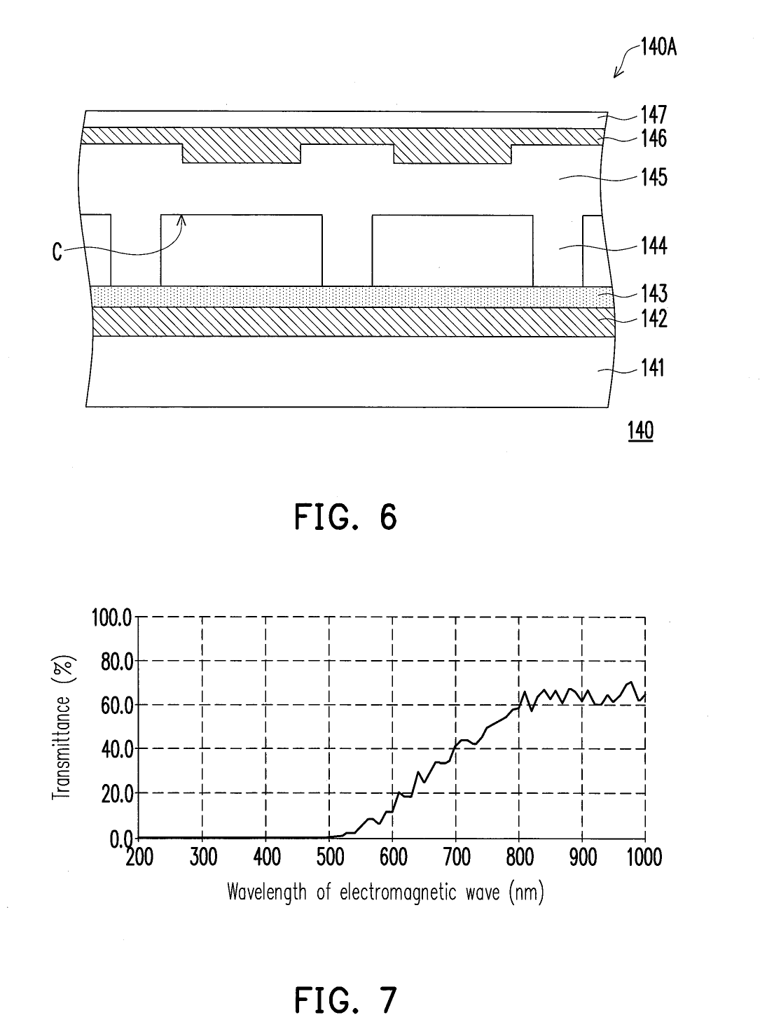

FIG. 6 is a partial cross-sectional view of the ultrasonic sensor in FIG. 1. Referring to FIG. 6, in the present embodiment, the ultrasonic sensor 140 includes a plurality of ultrasonic sensing units 140A. Each ultrasonic sensing unit 140A includes an electromagnetic wave transmissible substrate 141, a first electromagnetic wave transmissible electrode 142, an electromagnetic wave transmissible insulation layer 143, a patterned electromagnetic wave transmissible support structure 144, an electromagnetic wave transmissible film 145, and a second electromagnetic wave transmissible electrode 146. The first electromagnetic wave transmissible electrode 142 is disposed on the electromagnetic wave transmissible substrate 141. The electromagnetic wave transmissible insulation layer 143 is disposed on the first electromagnetic wave transmissible electrode 142. The patterned electromagnetic wave transmissible support structure 144 is disposed on the electromagnetic wave transmissible insulation layer 143. The electromagnetic wave transmissible film 145 is disposed on the patterned electromagnetic wave transmissible support structure 144. At least one cavity C is formed between the electromagnetic wave transmissible insulation layer 143, the patterned electromagnetic wave transmissible support structure 144, and the electromagnetic wave transmissible film 145. The cavity C is filled with air or any other suitable gas. In addition, the second electromagnetic wave transmissible electrode 146 is disposed on the electromagnetic wave transmissible film 145. When the ultrasonic wave W is transmitted to the ultrasonic sensor 140, it vibrates the electromagnetic wave transmissible film 145 in the ultrasonic sensing units 140A. The first electromagnetic wave transmissible electrode 142 and the second electromagnetic wave transmissible electrode 146 detect the vibration of the electromagnetic wave transmissible film 145 and accordingly generate an electrical signal. Thereby, the ultrasonic sensing units 140A convert the ultrasonic wave W into an electrical signal.

In the present embodiment, the electromagnetic wave transmissible film 145 and the patterned electromagnetic wave transmissible support structure 144 can let an electromagnetic wave having a wavelength between 10 nm and 2400 nm to pass through. To be specific, the electromagnetic wave transmissible film 145 and the patterned electromagnetic wave transmissible support structure 144 are made of at least one of a polymer material, Si, SiO₂, Si₃N₄, Al₂O₃, a monocrystal material, and other materials that can let an electromagnetic wave having a wavelength between 10 nm and 2400 nm to pass through. Aforementioned polymer material includes at least one of benzocyclobutene (BCB), polyimide (PI), SU8 photoresist, polydimethylsiloxane (PDMS), and other polymer materials.

Additionally, in the present embodiment, the first electromagnetic wave transmissible electrode 142 and the second electromagnetic wave transmissible electrode 146 are made of at least one of ITO and IZO. Moreover, in the present embodiment, the electromagnetic wave transmissible substrate 141 is a glass substrate or a polymer soft substrate. In the present embodiment, each ultrasonic sensing unit 140A further includes an electromagnetic wave transmissible passivation layer 147. The electromagnetic wave transmissible passivation layer 147 is disposed on the second electromagnetic wave transmissible electrode 146 for protecting the second electromagnetic wave transmissible electrode 146.

Below, the electromagnetic wave transmissibility of the ultrasonic sensing units 140A will be validated through an optical simulation. However, this optical simulation is not intended to limit the scope of the disclosure. Those having ordinary knowledge in the art should be able to set parameters of aforementioned components according to embodiments of the disclosure without departing the scope of the disclosure.

In the present optical simulation, the electromagnetic wave transmissible substrate 141 is simulated by a BK7 optical glass having a thickness of 500 μm, the first electromagnetic wave transmissible electrode 142 and the second electromagnetic wave transmissible electrode 146 are respectively simulated by an ITO film having a thickness of 0.1 μm, the gas in the cavity C is simulated by air having a thickness of 1 μm, the electromagnetic wave transmissible film 145 is simulated by a dielectric layer (for example, a SiO₂ film) having a thickness of 1 μm, and the electromagnetic wave transmissible passivation layer 147 is simulated by a dielectric layer (for example, a PI film) having a thickness of 0.1 μm. The refractivity of the BK7 optical glass adopted in the present optical simulation is 1.51184, and the extinction coefficient thereof is 0. The refractivity of the ITO film is 1.88, and the absolute value of the extinction coefficient thereof is 0.0056. The refractivity of air is 1, and the extinction coefficient thereof is 0. The refractivity of SiO₂ is 1.454, and the extinction coefficient thereof is 0. The refractivity of PI is 1.65, and the absolute value of the extinction coefficient thereof is 0.0056. The transmittance of the ultrasonic sensing units 140A obtained through an optical simulation with foregoing parameters is 76.399%. Namely, the ultrasonic sensing units 140A in the present embodiment have a high transmittance.

Below, the effect that the electromagnetic wave transmitting needles 130 improve the penetration depth of the electromagnetic wave L will be validated through an optical simulation. However, this optical simulation is not intended to limit the scope of the disclosure. Those having ordinary knowledge in the art should be able to set parameters of aforementioned components according to embodiments of the disclosure without departing the scope of the disclosure.

TABLE 1

| | | Thickness (μm) | Refractivity |
|---|---|---|---|
| Human Skin | Stratum Corneum | 20 | 1.5 |
| | Stratum Basale | 120 | 1.4 |
| | Melanin | 20 | 1.4 |
| | Dermis | 2000 | 1.35 |
| | Hypodermis | 3000 | 1.44 |

The thickness and refractivity of each layer of human skin are listed in foregoing table 1. By assuming that an electromagnetic wave W passes through a flap of human skin having the parameters listed in foregoing table 1 and performing an optical simulation by using the physical parameters of human skin listed in foregoing table 1, the transmittance of human skin with respect to the electromagnetic wave W of different wavelength is obtained, as shown in FIG. 7. As shown in FIG. 7, without the electromagnetic wave transmitting needles 130 in the present embodiment, the electromagnetic wave W cannot pass through human skin effectively, especially when the wavelength of the electromagnetic wave W is smaller than 500 μm.

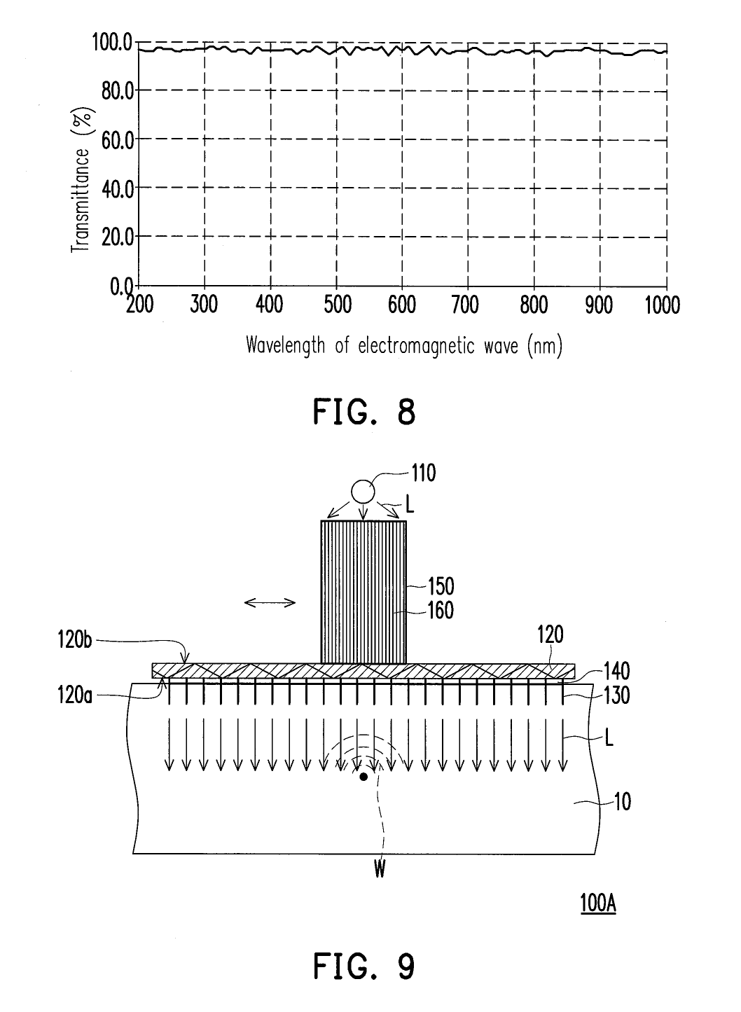

When the electromagnetic wave transmitting needles 130 in the present embodiment are adopted, the electromagnetic wave W can be considered directly entering the human skin from the dermis in table 1. By performing an optical simulation under foregoing condition, the transmittances of human skin to the electromagnetic wave W of different wavelengths are obtained, as shown in FIG. 8. It can be understood by comparing FIG. 7 and FIG. 8 that when the electromagnetic wave transmitting needles 130 in the present embodiment are adopted, the electromagnetic wave W can effectively pass through the exterior layers of the human skin and reach the interior layers thereof, so that the photoacoustic imaging apparatus 100 in the present embodiment can capture a photoacoustic image of high quality.

### Photoacoustic Image Capturing Method

Referring to FIG. 1 again, in the present embodiment, the method of capturing a photoacoustic image includes following steps. First, an object 10 is provided. Then, a first electromagnetic wave transmissible substrate 120 and a plurality of electromagnetic wave transmitting needles 130 disposed on the first electromagnetic wave transmissible substrate 120 are provided. Next, the first electromagnetic wave transmissible substrate 120 is laid on the object 10, and the electromagnetic wave transmitting needles 130 are inserted into the object 10. After that, an electromagnetic wave L is transmitted to the inside of the object 10 through the first electromagnetic wave transmissible substrate 120 and the electromagnetic wave transmitting needles 130. The inside of the object 10 generates an ultrasonic wave W in response to the electromagnetic wave. The ultrasonic wave W is then detected.

In the present embodiment, the step of detecting the ultrasonic wave W includes following steps. First, an ultrasonic sensor 140 is disposed on the transmission path of an electromagnetic wave L, wherein the ultrasonic sensor 140 is suitable for being passed through by the electromagnetic wave L, and the electromagnetic wave L is transmitted to the first electromagnetic wave transmissible substrate 120 after passing through the ultrasonic sensor 140. Next, the ultrasonic wave W is detected by using the ultrasonic sensor 140.

To be specific, in the present embodiment, the first electromagnetic wave transmissible substrate 120 has a first surface 120a and an opposite second surface 120b. The electromagnetic wave transmitting needles 130 is disposed on the first surface 120a. The step of detecting the ultrasonic wave W includes following steps. First, the ultrasonic sensor 140 is provided. Then, the ultrasonic sensor 140 is moved along the second surface 120b to detect the ultrasonic wave W.

### Second Embodiment

### Photoacoustic Imaging Apparatus

FIG. 9 is a cross-sectional view of a photoacoustic imaging apparatus according to the second embodiment of the disclosure. Referring to FIG. 9, the photoacoustic imaging apparatus 100A in the present embodiment is similar to the photoacoustic imaging apparatus 100 in the first embodiment and like reference numerals refer to like elements throughout. The difference between the photoacoustic imaging apparatus 100A in the present embodiment and the photoacoustic imaging apparatus 100 in the first embodiment falls on the position of the ultrasonic sensor 140. Below, this difference will be explained, while other similar aspects of the two embodiments will not be described again.

In the present embodiment, the first electromagnetic wave transmissible substrate 120 has a first surface 120a and an opposite second surface 120b. The electromagnetic wave transmitting needles 130 are disposed on the first surface 120a. The ultrasonic sensor 140 is disposed on the first surface 120a. The ultrasonic sensor 140 is disposed between the first electromagnetic wave transmissible substrate 120 and the object 10. The ultrasonic sensor 140 is suitable for being passed through by the electromagnetic wave L. The electromagnetic wave L passes through the ultrasonic sensor 140 to be transmitted to the inside of the object 10.

In other words, in the present embodiment, the ultrasonic sensor 140 and the electromagnetic wave transmitting needles 130 are all formed on the first surface 120a of the first electromagnetic wave transmissible substrate 120. When the electromagnetic wave transmitting needles 130 are inserted into the object 10, the ultrasonic sensor 140 contacts the object 10, so that the ultrasonic wave W emitted from the inside of the object 10 can reach the ultrasonic sensor 140 without passing through the first electromagnetic wave transmissible substrate 120. Thus, the photoacoustic imaging apparatus 100A in the present embodiment can capture a photoacoustic image of high quality.

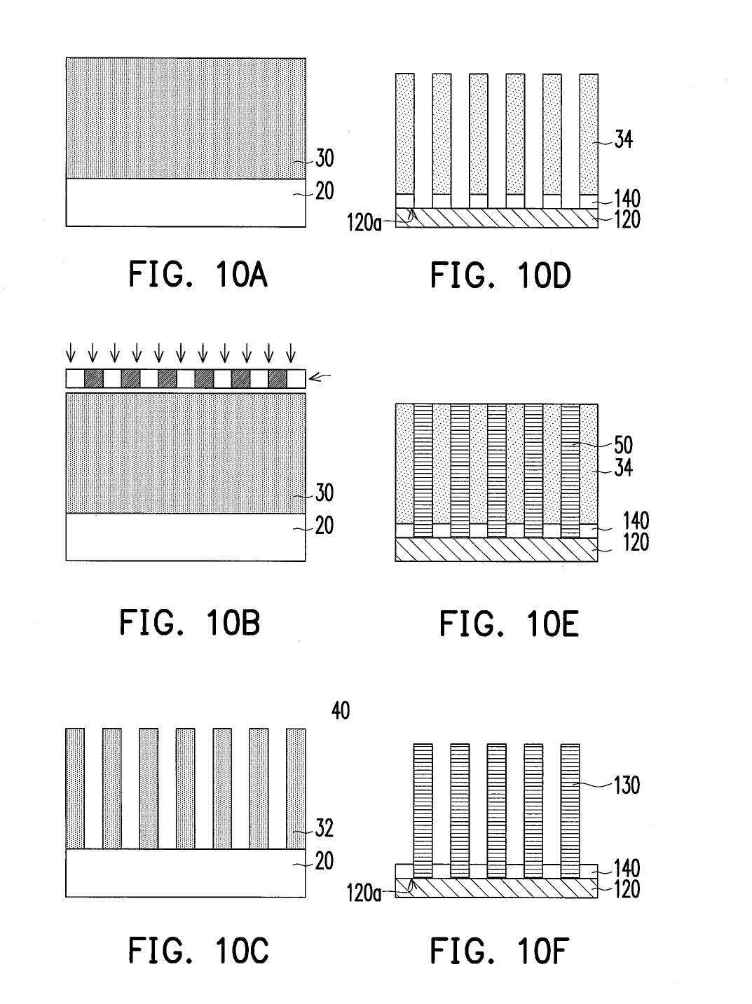

FIG. 10A˜FIG. 10F illustrates a process of integrating electromagnetic wave transmitting needles and an ultrasonic sensor onto a first electromagnetic wave transmissible substrate. Referring to FIG. 10A˜FIG. 10F, the ultrasonic sensor 140 is formed on the first surface 120a of the first electromagnetic wave transmissible substrate 120. Then, the electromagnetic wave transmitting needles 130 are formed on the first surface 120a through moulding. To be specific, referring to FIG. 10A, in the present embodiment, a polymer photosensitive material layer 30, such as BCB, PI, SU8, PDMS, etc., is first formed on a substrate 20. Referring to FIG. 10B˜FIG. 10C, then, a lithography process is performed on the polymer photosensitive material layer 30 by using a mask 40, so as to form a mould 32 for fabricating the electromagnetic wave transmitting needles 130. Referring to FIG. 10D, next, the mould 32 is moulded from a polydimenthylsiloxane (PDMS) mould 34. Next, the PDMS mould 34 is aligned with the ultrasonic sensor 140. Referring to FIG. 10E, a chitosan solution 50 is poured into the PDMS mould 34. Referring to FIG. 10F, The PDMS mould 34 is then parted to form the electromagnetic wave transmitting needles 130 formed by the chitosan solution 50, then the electromagnetic wave transmitting needles 130 formed in the ultrasonic sensor 140.

The function of the photoacoustic imaging apparatus 100A in the present embodiment is similar to that of the photoacoustic imaging apparatus 100 in the first embodiment therefore will not be described herein.

### Photoacoustic Image Capturing Method

Referring to FIG. 9 again, the method of capturing a photoacoustic image in the present embodiment includes following steps. First, an object 10 is provided. Then, a first electromagnetic wave transmissible substrate 120 and a plurality of electromagnetic wave transmitting needles 130 disposed on the first electromagnetic wave transmissible substrate 120 are provided. Next, the first electromagnetic wave transmissible substrate 120 is laid on the object 10, and the electromagnetic wave transmitting needles 130 are inserted into the object 10. An electromagnetic wave L is transmitted to the inside of the object 10 through the first electromagnetic wave transmissible substrate 120 and the electromagnetic wave transmitting needles 130. The inside of the object 10 generates an ultrasonic wave W in response to the electromagnetic wave L. The ultrasonic wave W is then detected.

Unlike that in the first embodiment, in the present embodiment, a user needs not to move the ultrasonic sensor 140 along the first surface 120a. To be specific, the step of detecting the ultrasonic wave W in the present embodiment includes following steps. First, an ultrasonic sensor 140 is provided. Then, the ultrasonic sensor 140 is fixed onto the first electromagnetic wave transmissible substrate 120 to cover the same. Next, the ultrasonic wave W is detected by using the ultrasonic sensor 140.

### Third Embodiment

### Photoacoustic Imaging Apparatus

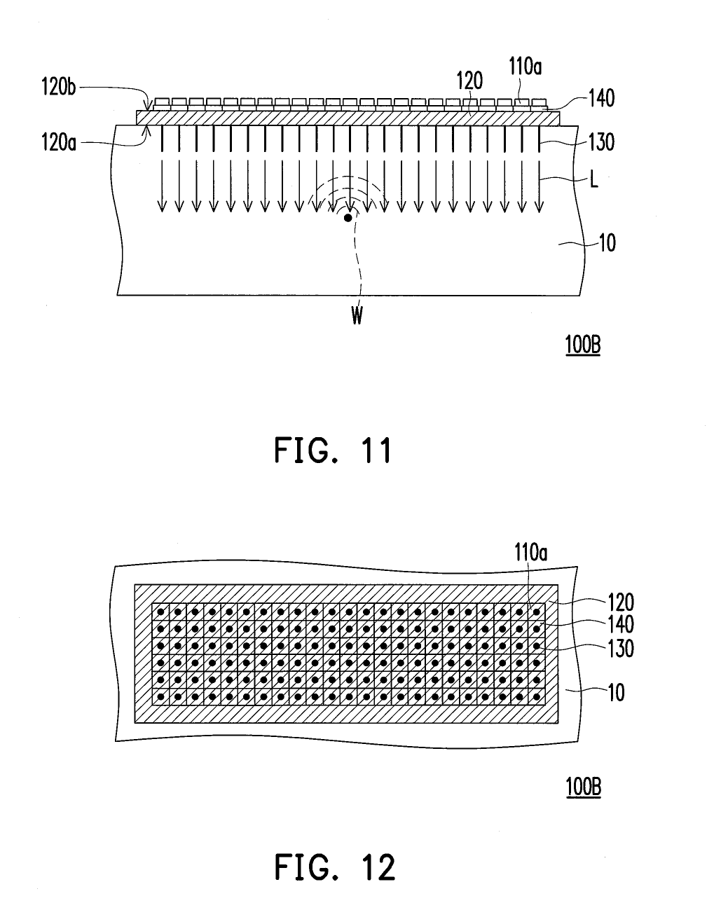

FIG. 11 is a cross-sectional view of a photoacoustic imaging apparatus according to the third embodiment of the disclosure. FIG. 12 is a top view of the photoacoustic imaging apparatus in FIG. 11. Referring to FIG. 11 and FIG. 12, the photoacoustic imaging apparatus 100B in the present embodiment is similar to the photoacoustic imaging apparatus 100A in the second embodiment, and like reference numerals refer to like elements throughout. The difference between the photoacoustic imaging apparatus 100B in the present embodiment and the photoacoustic imaging apparatus 100A in the second embodiment falls on the electromagnetic wave source 110 and the position thereof. Below, this difference will be explained, while other similar aspects of the two embodiments will not be described again.

In the present embodiment, the electromagnetic wave source is a plurality of electromagnetic wave source emitters 110a. The electromagnetic wave source emitters 110a may be laser diodes. The electromagnetic wave source emitters 110a are arranged on the first electromagnetic wave transmissible substrate 120 as an array. Besides, the ultrasonic sensor 140 is disposed between the electromagnetic wave source emitters 110a and the first electromagnetic wave transmissible substrate 120. Because the electromagnetic wave source emitters 110a are arranged on the first electromagnetic wave transmissible substrate 120 as an array, the electromagnetic wave source emitters 110a can supply a uniform and highly intensive electromagnetic wave L to the object 10 such that the performance of the photoacoustic imaging apparatus 100B in the present embodiment can be improved. In addition, the function of the photoacoustic imaging apparatus 100A in the present embodiment is similar to that of the photoacoustic imaging apparatus 100 in the first embodiment therefore will not be described again.

### Fourth Embodiment

### Photoacoustic Imaging Apparatus

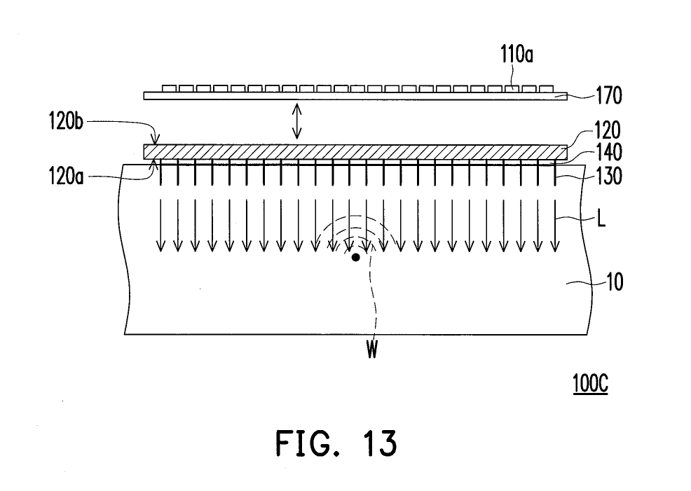

FIG. 13 is a cross-sectional view of a photoacoustic imaging apparatus according to the fourth embodiment of the disclosure. Referring to FIG. 13, the photoacoustic imaging apparatus 100C in the present embodiment is similar to the photoacoustic imaging apparatus 100B in the third embodiment and like reference numerals refer to like elements throughout. The difference between the photoacoustic imaging apparatus 100C in the present embodiment and the photoacoustic imaging apparatus 100A in the second embodiment is that the photoacoustic imaging apparatus 100C in the present embodiment further includes a second electromagnetic wave transmissible substrate 170 and the electromagnetic wave source emitters 110a is disposed on the second electromagnetic wave transmissible substrate 170. Below, this difference will be explained, while other similar aspects of the two embodiments will not be described again.

The photoacoustic imaging apparatus 100C in the present embodiment further includes a second electromagnetic wave transmissible substrate 170. The electromagnetic wave source emitters 110a are disposed on the second electromagnetic wave transmissible substrate 170. The second electromagnetic wave transmissible substrate 170 is mounted on the first electromagnetic wave transmissible substrate 120, and the second electromagnetic wave transmissible substrate 170 is between the electromagnetic wave source 110 and the first electromagnetic wave transmissible substrate 120. In other words, after capturing a photoacoustic image of the inside of the object 10, a user can separate the second electromagnetic wave transmissible substrate 170 and the first electromagnetic wave transmissible substrate 120 and discard the used first electromagnetic wave transmissible substrate 120 and electromagnetic wave transmitting needles 130, so as to avoid the risk of contagion. The remaining second electromagnetic wave transmissible substrate 170 and the electromagnetic wave source emitters 110a can be reused so that the cost of capturing photoacoustic images can be reduced. In addition, the function of the photoacoustic imaging apparatus 100C in the present embodiment is similar to that of the photoacoustic imaging apparatus 100B in the third embodiment therefore will not be described herein.

As described above, in an embodiment of the disclosure, a photoacoustic imaging apparatus can effectively transmit an electromagnetic wave to the inside of an object through electromagnetic wave transmitting needles, so that a high-quality photoacoustic image can be captured by the photoacoustic imaging apparatus.

It will be apparent to those skilled in the art that various modifications and variations can be made to the structure of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. A photoacoustic imaging apparatus, suitable for detecting a photoacoustic image of an object, the photoacoustic imaging apparatus comprising:
an electromagnetic wave source, suitable for emitting an electromagnetic wave;
a first electromagnetic wave transmissible substrate, disposed on a transmission path of the electromagnetic wave;
a plurality of electromagnetic wave transmitting needles, disposed on the first electromagnetic wave transmissible substrate and suitable for being inserted into the object, wherein the electromagnetic wave is transmitted to at least a part of the electromagnetic wave transmitting needles through the first electromagnetic wave transmissible substrate, and the electromagnetic wave is transmitted to an inside of the object through at least the part of the electromagnetic wave transmitting needles; and
an ultrasonic sensor, disposed at one side of the object, wherein the inside of the object generates an ultrasonic wave in response to the electromagnetic wave, and the ultrasonic sensor is capable detecting the ultrasonic wave.

2. The photoacoustic imaging apparatus according to claim 1, wherein the first electromagnetic wave transmissible substrate comprises a first surface and an electromagnetic wave incident surface, the electromagnetic wave transmitting needles are disposed on the first surface, the electromagnetic wave enters the first electromagnetic wave transmissible substrate through the electromagnetic wave incident surface, and the electromagnetic wave is transmitted into the electromagnetic wave transmitting needles dispersedly through the first surface under a guidance of the first electromagnetic wave transmissible substrate.

3. The photoacoustic imaging apparatus according to claim 1 further comprising an electromagnetic wave transmitter, wherein the electromagnetic wave transmitter is capable transmitting the electromagnetic wave from the electromagnetic wave source to the first electromagnetic wave transmissible substrate.

4. The photoacoustic imaging apparatus according to claim 3, wherein the electromagnetic wave transmitter is a fiber bundle.

5. The photoacoustic imaging apparatus according to claim 3, wherein the electromagnetic wave transmitter has an electromagnetic wave exit surface, the ultrasonic sensor is disposed on the electromagnetic wave exit surface, the ultrasonic sensor is suitable for being passed through by the electromagnetic wave, and the electromagnetic wave transmitted in the electromagnetic wave transmitter sequentially passes through the electromagnetic wave exit surface and the ultrasonic sensor to be transmitted to the first electromagnetic wave transmissible substrate.

6. The photoacoustic imaging apparatus according to claim 3, wherein the ultrasonic sensor surrounds the electromagnetic wave transmitter.

7. The photoacoustic imaging apparatus according to claim 3, wherein the electromagnetic wave transmitter surrounds the ultrasonic sensor.

8. The photoacoustic imaging apparatus according to claim 1, wherein the first electromagnetic wave transmissible substrate has a first surface and a second surface that are opposite to each other, the electromagnetic wave transmitting needles are disposed on the first surface, the ultrasonic sensor is disposed on the first surface or the second surface, the ultrasonic sensor is suitable for being passed through by the electromagnetic wave, and the electromagnetic wave passes through the ultrasonic sensor to be transmitted to the inside of the object.

9. The photoacoustic imaging apparatus according to claim 8, wherein the electromagnetic wave source is a plurality of electromagnetic wave source emitters, and the electromagnetic wave source emitters are arranged on the first electromagnetic wave transmissible substrate as an array.

10. The photoacoustic imaging apparatus according to claim 8, wherein the ultrasonic sensor is between the electromagnetic wave source emitters and the first electromagnetic wave transmissible substrate.

11. The photoacoustic imaging apparatus according to claim 8 further comprising a second electromagnetic wave transmissible substrate, wherein the electromagnetic wave source is disposed on the second electromagnetic wave transmissible substrate, the second electromagnetic wave transmissible substrate is mounted on the first electromagnetic wave transmissible substrate, and the second electromagnetic wave transmissible substrate is between the electromagnetic wave source and the first electromagnetic wave transmissible substrate.

12. The photoacoustic imaging apparatus according to claim 11, wherein the electromagnetic wave source is a plurality of electromagnetic wave source emitters, and the electromagnetic wave source emitters are arranged on the second electromagnetic wave transmissible substrate as an array.

13. The photoacoustic imaging apparatus according to claim 1, wherein the ultrasonic sensor comprises a plurality of ultrasonic sensing units, and each of the ultrasonic sensing units comprises:
an electromagnetic wave transmissible substrate;
a first electromagnetic wave transmissible electrode, disposed on the electromagnetic wave transmissible substrate;
an electromagnetic wave transmissible insulation layer, disposed on the first electromagnetic wave transmissible electrode;
a patterned electromagnetic wave transmissible support structure, disposed on the electromagnetic wave transmissible insulation layer;
an electromagnetic wave transmissible film, disposed on the patterned electromagnetic wave transmissible support structure, wherein at least one cavity is formed between the electromagnetic wave transmissible insulation layer, the patterned electromagnetic wave transmissible support structure, and the electromagnetic wave transmissible film; and
a second electromagnetic wave transmissible electrode, disposed on the electromagnetic wave transmissible film.

14. The photoacoustic imaging apparatus according to claim 13, wherein a material of the electromagnetic wave transmissible film and the patterned electromagnetic wave transmissible support structure comprises at least one of a polymer material, Si, quartz, SiO₂, Si₃N₄, Al₂O₃, and a monocrystal material.

15. The photoacoustic imaging apparatus according to claim 13, wherein a material of the first electromagnetic wave transmissible electrode and the second electromagnetic wave transmissible electrode comprises at least one of ITO and IZO.

16. The photoacoustic imaging apparatus according to claim 1, wherein a length of the electromagnetic wave transmitting needles falls within a range of 100 μm to 1000 μm.

17. The photoacoustic imaging apparatus according to claim 1, wherein a diameter of the electromagnetic wave transmitting needles falls within a range of 20 μm to 300 μm.

18. The photoacoustic imaging apparatus according to claim 1, wherein a material of the electromagnetic wave transmitting needles is biocompatible.

19. The photoacoustic imaging apparatus according to claim 18, wherein the material of the electromagnetic wave transmitting needles is chitosan.

20. The photoacoustic imaging apparatus according to claim 1, wherein the first electromagnetic wave transmissible substrate has an ultrasonic wave impedance matching characteristic with respect to the object.

21. The photoacoustic imaging apparatus according to claim 1, wherein the first electromagnetic wave transmissible substrate is a flexible substrate.

22. The photoacoustic imaging apparatus according to claim 21, wherein a material of the first electromagnetic wave transmissible substrate comprises polyethylene terephthalate (PET) or polyimide.

23. The photoacoustic imaging apparatus according to claim 1, wherein the electromagnetic wave source is a laser generator.

24. The photoacoustic imaging apparatus according to claim 1, wherein a wavelength of the electromagnetic wave falls within a range of 10 nm to 2400 nm.

25. The photoacoustic imaging apparatus according to claim 1, wherein a transmittance of the ultrasonic sensor with respect to the electromagnetic wave is greater than 60%.

26. The photoacoustic imaging apparatus according to claim 1 further comprising a probe, wherein the electromagnetic wave is transmitted to the first electromagnetic wave transmissible substrate through the probe, the probe comprises an opening, and the electromagnetic wave is transmitted to the first electromagnetic wave transmissible substrate through the opening.

27. The photoacoustic imaging apparatus according to claim 26, wherein the opening is a linear opening, a circular opening, or an array-like opening.

28. A photoacoustic sensing structure, suitable for guiding an electromagnetic wave to an inside of an object to receive an ultrasonic wave generated by the inside of the object in response to the electromagnetic wave, the photoacoustic sensing structure comprising:
a first electromagnetic wave transmissible substrate, disposed on a transmission path of the electromagnetic wave;
a plurality of electromagnetic wave transmitting needles, disposed on the first electromagnetic wave transmissible substrate and suitable for being inserted into the object, wherein the electromagnetic wave is transmitted to at least a part of the electromagnetic wave transmitting needles through the first electromagnetic wave transmissible substrate, and the electromagnetic wave is transmitted to the inside of the object through at least the part of the electromagnetic wave transmitting needles; and
an ultrasonic sensor, disposed at one side of the object, wherein the inside of the object generates an ultrasonic wave in response to the electromagnetic wave, and the ultrasonic sensor detects the ultrasonic wave.

29. The photoacoustic sensing structure according to claim 28, wherein the first electromagnetic wave transmissible substrate comprises a first surface and an electromagnetic wave incident surface, the electromagnetic wave transmitting needles are disposed on the first surface, the electromagnetic wave enters the first electromagnetic wave transmissible substrate through the electromagnetic wave incident surface, and the electromagnetic wave is transmitted into the electromagnetic wave transmitting needles dispersedly through the first surface under a guidance of the first electromagnetic wave transmissible substrate.

30. The photoacoustic sensing structure according to claim 28, wherein the first electromagnetic wave transmissible substrate has a first surface and a second surface that are opposite to each other, the electromagnetic wave transmitting needles are disposed on the first surface, the ultrasonic sensor is disposed on the first surface or the second surface, the ultrasonic sensor is suitable for being passed through by the electromagnetic wave, and the electromagnetic wave passes through the ultrasonic sensor to be transmitted to the inside of the object.

31. The photoacoustic sensing structure according to claim 28, wherein the ultrasonic sensor comprises a plurality of ultrasonic sensing units, and each of the ultrasonic sensing units comprises:
an electromagnetic wave transmissible substrate;
a first electromagnetic wave transmissible electrode, disposed on the electromagnetic wave transmissible substrate;
an electromagnetic wave transmissible insulation layer, disposed on the first electromagnetic wave transmissible electrode;
a patterned electromagnetic wave transmissible support structure, disposed on the electromagnetic wave transmissible insulation layer;
an electromagnetic wave transmissible film, disposed on the patterned electromagnetic wave transmissible support structure, wherein at least one cavity is formed between the electromagnetic wave transmissible insulation layer, the patterned electromagnetic wave transmissible support structure and the electromagnetic wave transmissible film; and
a second electromagnetic wave transmissible electrode, disposed on the electromagnetic wave transmissible film.

32. The photoacoustic sensing structure according to claim 31, wherein a material of the electromagnetic wave transmissible film and the patterned electromagnetic wave transmissible support structure comprises at least one of a polymer material, Si, quartz, SiO₂, Si₃N₄, Al₂O₃, and a monocrystal material.

33. The photoacoustic sensing structure according to claim 31, wherein a material of the first electromagnetic wave transmissible electrode and the second electromagnetic wave transmissible electrode comprises at least one of ITO and IZO.

34. The photoacoustic sensing structure according to claim 28, wherein a length of the electromagnetic wave transmitting needles falls within a range of 100 μm to 1000 μm.

35. The photoacoustic sensing structure according to claim 28, wherein a diameter of the electromagnetic wave transmitting needles falls within a range of 20 μm to 300 μm.

36. The photoacoustic sensing structure according to claim 28, wherein a material of the electromagnetic wave transmitting needles is biocompatible.

37. The photoacoustic sensing structure according to claim 36, wherein the material of the electromagnetic wave transmitting needles is chitosan.

38. The photoacoustic sensing structure according to claim 28, wherein the first electromagnetic wave transmissible substrate has an ultrasonic wave impedance matching characteristic with respect to the object.

39. The photoacoustic sensing structure according to claim 28, wherein the first electromagnetic wave transmissible substrate is a flexible substrate.

40. The photoacoustic sensing structure according to claim 39, wherein a material of the first electromagnetic wave transmissible substrate comprises PET or polyimide.

41. The photoacoustic sensing structure according to claim 28, wherein a transmittance of the ultrasonic sensor with respect to the electromagnetic wave is greater than 60%.

42. A method of capturing a photoacoustic image, comprising:
providing an object;
providing a first electromagnetic wave transmissible substrate and a plurality of electromagnetic wave transmitting needles disposed on the first electromagnetic wave transmissible substrate;
laying the first electromagnetic wave transmissible substrate on the object, and inserting the electromagnetic wave transmitting needles into the object;
transmitting an electromagnetic wave to an inside of the object through the first electromagnetic wave transmissible substrate and at least a part of the electromagnetic wave transmitting needles, wherein the inside of the object generates an ultrasonic wave in response to the electromagnetic wave; and
detecting the ultrasonic wave.

43. The method according to claim 42, wherein the first electromagnetic wave transmissible substrate has a first surface and a second surface that are opposite to each other, the electromagnetic wave transmitting needles are disposed on the first surface, and the step of detecting the ultrasonic wave comprises:
providing an ultrasonic sensor; and
moving the ultrasonic sensor on the second surface to detect the ultrasonic wave.

44. The method according to claim 42, wherein the step of detecting the ultrasonic wave comprises:
providing an ultrasonic sensor;
covering and fixing the ultrasonic sensor onto the first electromagnetic wave transmissible substrate; and
detecting the ultrasonic wave by using the ultrasonic sensor.

45. The method according to claim 42, wherein the step of detecting the ultrasonic wave comprises:
disposing an ultrasonic sensor on a transmission path of the electromagnetic wave, wherein the ultrasonic sensor is suitable for being passed through by the electromagnetic wave, and the electromagnetic wave is transmitted to the first electromagnetic wave transmissible substrate after passing through the ultrasonic sensor; and
detecting the ultrasonic wave by using the ultrasonic sensor.

46. The method according to claim 42, wherein the first electromagnetic wave transmissible substrate has an ultrasonic wave impedance matching characteristic with respect to the object.

47. The method according to claim 42, wherein a wavelength of the electromagnetic wave falls within a range of 10 nm to 2400 nm.

48. The method according to claim 42, wherein a transmittance of an ultrasonic sensor with respect to the electromagnetic wave is greater than 60%.

49. The method according to claim 42, wherein the object is a creature's skin.
